(19) United States
(12) Patent Application Publication
(10) Pub. No.: US 2021/0181108 A1
(43) Pub. Date: Jun. 17, 2021

(54) METHOD OF MONITORING A FLUID AND USE OF A TRACER FOR MONITORING A FLUID

(71) Applicant: JOHNSON MATTHEY PUBLIC LIMITED COMPANY, London (GB)

(72) Inventors: Thomas William BURNS, Billingham (GB); Felicity Jane ROBERTS, Billingham (GB)

(21) Appl. No.: 17/269,187

(22) PCT Filed: Aug. 8, 2019
(86) PCT No.: PCT/GB2019/052230

§ 371 Date: Feb. 22, 2021

(30) Foreign Application Priority Data
Aug. 28, 2018 (GB) 1813976.6

(51) Int. Cl. G01N 21/64; E21B 49/08; G01N 33/28

(52) U.S. Cl. CPC G01N 21/643; E21B 49/086; G01N 2021/6439; G01N 33/2823; E21B 49/088

(57) ABSTRACT

A method of monitoring a fluid is described comprising introducing a luminescent nanoparticle into the fluid; removing a fluid sample from the fluid; adding a reagent to the fluid sample to vary the luminescence behaviour of the luminescent nanoparticles and/or of other luminescent species present in the fluid; analysing the luminescence of the modified fluid sample to determine an amount of the nanoparticle present therein. A use of a tracer based on these principles is also described.

### FIELD OF THE INVENTION

The present invention relates to methods of monitoring of fluids and to the use of nanoparticle tracers in monitoring of fluids. The invention in particular applies to the use of nanoparticle tracers in monitoring of hydrocarbon wells, pipelines or formations, but may also find application in process diagnostics and other areas where the use of a tracer or taggant composition may be applicable. More specifically, but not exclusively, the invention relates to the use of nanoparticle tracers for monitoring fluids produced and injected water from different zones of hydrocarbon wells and to methods of monitoring the same.

### BACKGROUND

The use of tracers to monitor aspects of the performance of hydrocarbon wells is an established technique. The tracers may be water tracers, in that they are predominantly soluble or dispersible in water, produced water, or water-based fluids; or may be oil tracers, in that they are soluble or dispersible in hydrocarbons or oil-based fluids in the formation; or partitioning tracers, in that they are soluble or dispersible between both the water and hydrocarbon phases. Some tracing methods will employ more than one type of tracer and use the difference in behaviour to deduce properties of the hydrocarbon formation. For example, partitioning and water tracers may be injected into a production well along with injected water and then monitored as they are subsequently produced from the well. The time difference between the production of the water tracers, which are produced with the returning injected water, and the partitioning tracers, whose production is delayed by their interaction with the hydrocarbons in the formation, can be used to deduce parameters relating to the local remaining hydrocarbon content of the formation. Alternatively, applications may use only water tracers. For example, water tracers may be introduced in an injection well and their presence monitored at adjacent production wells to obtain information about the flux of water from the injection well to the production well. In addition to injected techniques, it is also known to introduce tracers into a well by including them in articles placed into the well. By detecting the rate of tracer production over time, information can be deduced about performance of the hydrocarbon well.

To be useful as a tracer, a compound should be thermally stable in that it should be stable at the temperatures typically encountered in hydrocarbon wells, which may be 60 to 90° C. Desirably, a tracer is stable in temperatures up to maybe 160 or 180° C. so as to permit use in high temperature hydrocarbon wells. For a water tracer, the compound should be highly selective toward water over oil and will preferentially disperse in water over oil. The compound should also be detectable in small to very small quantities, for example at levels below 100 ppb, preferably at levels of 50 ppb or lower, more preferably at levels of 10 ppb or lower, and most preferably in the parts per trillion (ppt) range (that is, at levels less than 1 ppb). The levels are determined on a mass/mass basis. The compound should also be environmentally acceptable with low toxicity, for inserting into the ground, but also not a compound that is naturally present in the ground in such quantities as to contaminate the results of the tracer study.

Typical detection methods include gas chromatography-mass spectrometry (GC-MS), gas chromatography-mass spectrometry-mass spectrometry (GC-MS-MS), liquid chromatography-mass spectrometry (LC-MS), liquid chromatography-mass spectrometry-mass spectrometry (LC-MS-MS) and high-pressure liquid chromatography (HPLC), which can typically detect very low concentrations of the tracers in the produced fluids. It is desirable that tracers should be detectable in low quantities and also that they can be reliably distinguished from other tracers.

Tracers may comprise or include a luminophore (that is, a material that can emit energy upon excitation with energy) and a presence of the tracer may be determined by optical spectroscopy of that emission. Known luminophores include fluorophores, that is, materials that exhibit fluorescence. However, known fluorophores may have undesirable properties relating to their performance as tracers or to their environmental impact. For example, known fluorophores are based on metallic quantum dots but these are frequently toxic or environmentally damaging and as a result tend to require an inert coating such as a silica coating. For example, known fluorophores are based on fluorescent dyes but these tend to be unstable in hydrocarbon well conditions.

Luminescent and for example fluorescent nanoparticles have attracted recent attention for such application. Examples include semiconductor quantum dots, metallic quantum dots, carbon dots, and other carbon-based nanomaterials.

Produced fluid from hydrocarbon wells generally contains organic species which are naturally fluorescent. This fluorescence tends to be exhibited at the blue end of the visible spectrum, overlapping with that of carbon-based nanoparticles dots such as are described in U.S. Pat. No. 9,891,170, which exhibit a peak fluorescence intensity occurring at an emission wavelength of for example 440 to 475 nm. This may limit the effective use of such carbon dots without significant fluid preparation, separation and cleaning, such as is described in U.S. Pat. No. 9,891,170.

These organic species which are naturally fluorescent in the relevant wavelength range are not limited to the oil phase. Some of the organic species exhibit appreciable water solubility and may be present in produced water. As a consequence, even complete separation of oil and water phases in the produced fluid will not prevent this effect.

Metallic quantum dots are known that fluoresce at longer wavelengths and are less likely to overlap with the fluorescence wavelengths of residual organics but these are typically based on heavy metals such as lead and cadmium and their use as tracers would raise environmental issues. These materials are also known to be susceptible to photobleaching.

Methods of enhancing the fluorescence of nanoparticles for use in such applications are therefore desirable. The present invention seeks to overcome one or more of the above disadvantages of the prior art and in particular to provide such enhanced fluorescence. In particular, preferred embodiments of the present invention seek to provide improved carbon-based nanoparticle tracers for use in hydrocarbon well monitoring and in particular for use in monitoring produced water from hydrocarbon wells.

### SUMMARY OF INVENTION

According to a first broadest aspect of the invention, there is provided a method of monitoring an aqueous fluid, comprising:

introducing a luminescent nanoparticle into the fluid; removing a fluid sample from the fluid;
adding a reagent to the fluid sample to vary the luminescence behaviour of the luminescent nanoparticles and/or of other luminescent species present in the fluid;
analysing the luminescence of the modified fluid sample to determine an amount of the nanoparticle present therein.

The invention thus comprises a method of monitoring an aqueous fluid based on the use of luminophores, wherein the luminophores comprise luminescent nanoparticles. In the context of this invention a luminophore is a material that emits light by luminescence (that is, a material that can emit light upon excitation) by a mechanism that may include without limitation fluorescence and phosphorescence.

Luminophores based on a fluorescence mechanism, and therefore fluorescent nanoparticles, are particularly preferred. Where reference is consequently made herein to fluorescence and to fluorescent nanoparticles by way of example, the skilled person will nevertheless understand that unless the context demands to the contrary, the principles may be applied to other mechanisms of luminescence provided that the necessary variation with reagent addition is exhibited by the luminescent nanoparticle.

The invention thus comprises a method of monitoring an aqueous fluid by introducing luminescent nanoparticles for example as tracers and batch sampling of the fluid to draw inferences about the fluid based on an analysed absence, presence or presence at a particular level of the luminescent nanoparticle in the sample. Such principles of fluid monitoring are generally known.

The method is particularly directed at the monitoring of aqueous fluids that might be expected to contain organic species which are naturally fluorescent. Examples include, but the invention is not limited to, produced fluids from hydrocarbon wells. The method is distinctly characterised by the step of introducing a reagent to the fluid sample to vary the luminescence behaviour of the luminescent nanoparticles and/or of those other luminescent species present in the fluid so as to enable the better resolution of the luminescence of the luminescent nanoparticles from the background luminescence of the other species therein.

More particularly, the reagent will be selected to act either to suppress the luminescence of the other species therein or to increase the luminescence of the luminescent nanoparticles so as to enable the better resolution of the luminescence of the luminescent nanoparticles from the background luminescence of the other species therein.

Typical luminescence testing will be carried out at one or more specific wavelengths, and for example fluorescence testing at one or more specific excitation wavelengths. It will be understood that references herein to analysing the luminescence will in such cases be luminescence at the said specific wavelength or wavelengths. It will be understood that references to suppression/increase of luminescence will in such cases be references to suppression/increase of luminescence at the said specific wavelength or wavelengths.

In a preferred embodiment, the method comprises adding the reagent at least to vary the luminescence of the luminescent nanoparticles. In such a case, the method comprises first selecting a combination of luminescent nanoparticles and reagent that are known to interact together in aqueous solution such that the luminescence behaviour of the luminescent nanoparticles varies in the presence of the reagent.

A reagent may be selected to vary the luminescence behaviour of the luminescent nanoparticles and/or of those other luminescent species present in the fluid by any suitable physical or chemical mechanism.

For example, the reagent may be selected that interacts preferentially and for example reacts chemically with the luminescent nanoparticles or with at least some other luminescent species present in such a manner that the said interaction affects the luminescence of the same.

For example, the reagent may change a condition parameter of the fluid, the said condition parameter being one the variation of which is known to cause a variation in luminescence of the luminescent nanoparticles and/or of those other luminescent species present in the fluid. In such a case, the method preferably comprises the additional step of measuring a condition parameter of the fluid sample before adding the reagent, and subsequently adding the reagent to change the condition parameter in such manner as to tend to suppress the luminescence of the other species in the fluid sample or to increase the luminescence of the luminescent nanoparticles.

One or more further reagents may be added in addition to the said reagent as a first reagent. In such a case, at least the first reagent is selected to vary the luminescence behaviour of the luminescent nanoparticles and/or of other luminescent species present in the fluid. The further reagent(s) may similarly be selected to vary the luminescence behaviour of the luminescent nanoparticles and/or of other luminescent species present in the fluid. Additionally or alternatively, at least one further reagent may be selected to otherwise enhance the detectability of the tracer in the fluid sample. Additionally or alternatively, at least one further reagent may be selected to otherwise modify the fluid sample, for example to stabilise the nanoparticles therein.

In a preferred case, a reagent may change a condition parameter of the fluid. A particularly preferred example of such a condition parameter is the pH of the aqueous fluid. Another preferred example of such a condition parameter is the ionic strength of the aqueous fluid.

Certain classes of fluorescent nanoparticle may be selected or fabricated to exhibit a luminescence that varies with pH, and in particular that exhibits an intensity at a particular excitation wavelength that changes with pH.

Thus, in a particular embodiment, the method comprises: selecting a luminescent nanoparticle known to undergo luminescence that exhibits an intensity in an aqueous fluid that varies with pH;

introducing the nanoparticle into the fluid;
removing a fluid sample from the fluid;
determining a pH of the fluid;
adding a reagent to modify the pH of the fluid sample;
analysing the luminescence of the modified fluid sample to determine an amount of the nanoparticle present therein.

The particular embodiment of the method is characterised in two ways.

First, the nanoparticle to be introduced is selected from the sub-class of luminescent nanoparticles that exhibits a variable luminescence response with pH, and in the preferred case the nanoparticle is selected to be one that exhibits a variable fluorescence response with pH and for example a maximum fluorescence intensity at a particular pH.

Second, between taking of a sample from the fluid and testing of the sample for presence of luminescent nanoparticles a reagent is added to modify the pH of the sample. In particular for example, the pH may be modified in accordance with the invention in such manner as to tend to move the pH of the sample from an unmodified pH to a modified pH, where the luminescence and for example the luminescent nanoparticle exhibits a more intense luminescence at the modified pH than at the unmodified pH.

Preferably the luminescence and for example fluorescence of the nanoparticle is at least 10% higher at the modified pH than at the unmodified pH. It follows that preferably the nanoparticle is selected to exhibit a variable luminescence response of such an extent across a pH range that represents a range that can be practically modified by addition of a suitable reagent.

In a possible case, the nanoparticle is selected to exhibit a variable luminescence and for example fluorescence response with pH having a maximum luminescence intensity at a particular pH, and the pH is modified in such manner as to tend to move the pH of the sample from an unmodified pH to a modified pH, where the modified pH is closer than the unmodified pH to the pH of maximum luminescence.

The invention in the preferred embodiment thus comprises selecting a luminescent nanoparticle tracer known to undergo luminescence that exhibits an intensity in an aqueous fluid that varies with pH. Such an effect may be provided or enhanced in that the luminescent nanoparticle comprises a coating and/or a surface modification such as to cause the nanoparticle to exhibit a luminescence response that varies with varying pH.

Advantageously, additionally the luminescence of certain of the other species present in a typical solution may be found to vary with pH. With careful selection of the properties of nanoparticle and reagent it may be possible in an ideal case simultaneously to enhance the luminescence of nanoparticles and to suppress of at least some of the background luminescence.

Again, it will be understood that references to analysing, increasing or suppressing luminescence or fluorescence with pH include analysing, increasing or suppressing luminescence or fluorescence, can be measured at one or more particular wavelengths.

Additionally, or alternatively, the luminescence intensity of the luminescent nanoparticle may be modified by doping.

Although a variation in fluorescence with pH in aqueous solutions has been observed as a theoretical property in some fluorescent nanoparticles, there are various difficulties in exploiting this in practical systems.

For example, known fluorophores based on metallic quantum dots might in principle exhibit such a property but their toxic and/or environmentally damaging nature tends to require an inert coating such as a silica coating which would tend to suppress such effects. Fluorophores based on fluorescent dyes tend to be unstable and are susceptible to photobleaching. This would tend to mandate use in stable environments and militate against any method that involved the addition of secondary reagents to vary pH excessively.

However, in accordance with the principles of the invention nanoparticles may be selected in which these disadvantages are mitigated.

In a preferred case, a nanoparticle for use in accordance with the invention comprises a carbon-based nanoparticle. Carbon-based nanoparticles (for example less than 100 nm in size, and generally less than 10 nm in size) have been found to exhibit useful luminescent properties and in particular fluorescent properties together with low toxicity, high chemical stability and less susceptible to photobleaching that make them potentially attractive for tracer applications. Carbon-based nanoparticles are also known in the literature as carbon quantum dots, C-dots, carbon nanoparticles, carbon dots, amorphous carbon dots, graphitic carbon dots, graphene quantum dots or graphene dots. Novel carbon quantum dot (CAD-) based fluorescent tracers have been proposed for production and well monitoring. They may be structured or have surface modifications to exhibit high dispersibility in water. Their use as aqueous phase tracers has been discussed for example in U.S. Pat. No. 9,891,170.

In a preferred case, the method comprises selecting a carbon-based nanoparticle and in particular a carbon-based luminescent nanoparticle and for example a carbon-based fluorescent nanoparticle.

Carbon-based nanoparticles are considered to emit light by fluorescence, and references to emission of light or luminescence may accordingly in this context be construed to be references to fluorescence.

Carbon-based nanoparticles are particularly suited to the method of the present invention as they can readily be given functionalized coatings or surface modifications which may both enhance their chemical stability in a range of environments and especially a range of pH conditions and cause them to exhibit a luminescence response that varies significantly in the presence of various reagents/in particular conditions, and most particularly that varies significantly with varying pH.

In a preferred case, the method comprises selecting a fluorescent carbon-based nanoparticle which has a coating and/or a surface modification such as to cause the nanoparticle to exhibit a fluorescence response that varies in the presence of various reagents/in particular conditions, and most particularly that varies with varying pH.

For example, the surface may be modified by the presence of one or more functional groups that act in aqueous solution as proton donors or proton acceptors. The provision of such functional groups will tend to cause the nanoparticle to exhibit a fluorescence response that varies in the presence of various reagents/in particular conditions, and most particularly that varies with varying pH.

Suitable functional groups may be selected from: carboxyl, carbonyl, sulfonyl, hydroxyl, thiol, amine, amide, imide, in their protonated or unprotonated forms, and combinations and derivatives of the same.

Carbon-based nanoparticles are particularly suited to the provision of such functionalised coatings and/or surface modifications, but the principle of creating or enhancing the required effect of a luminescence response that varies significantly with varying pH may be applied to any luminescent nanoparticles for application in accordance with the invention.

In the preferred case of the invention where carbon-based nanoparticles such as exemplified above are used as luminescent nanoparticles, any suitable fabrication technique may be used for their manufacture. The fabrication of the carbon-based nanoparticles is generally either by the breaking down of larger carbonaceous structures such as nanodiamonds, graphite, carbon nanotubes, graphene sheets, carbon soot and the like by methods including arc discharge, laser ablation, sonication, chemical ablation, electrochemical carbonization and microwave irradiation; or by synthesis from molecular precursors by methods including combustion/thermal treatments, supported synthetic, sonication and microwave synthetic routes etc.

A known method of forming carbon-based nanoparticles suitable for use in accordance with the invention is to provide an electrochemical cell including at least one graphite electrode and an electrolyte which may comprise another unique carbon source. A current is applied across electrodes of the electrochemical cell to form carbon-based nanoparticles comprising carbon from the carbon source.

Another known method of forming carbon-based nanoparticles suitable for use in accordance with the invention is to make use of microwave irradiation to thermally heat a solution of molecular precursors.

Another known method of forming carbon-based nanoparticles suitable for use in accordance with the invention is to make use of a hydrothermal or solvothermal technique to heat a solution of molecular precursors.

In the preferred case where carbon-based nanoparticles are used as luminescent nanoparticles, the carbon-based nanoparticles may be doped. A large range of potential dopants is available. The carbon-based nanoparticles may be doped by addition of one or more metal species. The carbon-based nanoparticles may be doped by addition of one or more non-metallic species. For example, the carbon-based nanoparticles may be doped by addition of one or more of nitrogen, sulfur, boron, silicon, fluorine, selenium, titanium, magnesium, bismuth and phosphorus to form nitrogen-doped, sulfur-doped, boron-doped, silicon-doped, fluorine-doped, selenium-doped, titanium-doped, magnesium-doped, bismuth-doped and phosphorus-doped carbon-based nanoparticles, respectively. Techniques for preparing carbon-based nanoparticles with such dopants are known.

A particular preferred use of the method according to the first aspect of the invention is use in monitoring a parameter of a hydrocarbon well, pipeline or formation, and discussion herein considers such use by way of example, but other uses for example in process diagnostics and other areas where the use of a tracer or taggant composition may be encompassed within the scope of the invention.

It follows that a preferred embodiment of the method according to the first aspect of the invention comprises a method of monitoring of a parameter of a hydrocarbon well, pipeline or formation, the method comprising:

In the preferred embodiment where a luminescent nanoparticle known to undergo luminescence that exhibits an intensity in an aqueous fluid that varies with pH is used and the reagent varies the pH, the method comprises:

selecting a luminescent nanoparticle known to undergo luminescence that exhibits an intensity in an aqueous fluid that varies with pH;
introducing the nanoparticle into the hydrocarbon well, pipeline or formation;
producing a fluid from the hydrocarbon well, pipeline or formation;
removing a fluid sample from the fluid;
adding a reagent to modify the pH of the fluid sample; analysing the modified fluid sample to determine an amount of the nanoparticle present therein.

However, the method of the first aspect of the invention may find other application for example in process diagnostics and other areas where the use of a tracer or taggant composition may be useful.

The step of determining the amount of luminescent nanoparticle present in the fluid encompasses either determining whether a luminescent nanoparticle is present or determining a quantity or proportion of the luminescent nanoparticle present or both.

Preferably the luminescent nanoparticle is selected to have utility as and is used as a water tracer. The fluid produced may therefore comprise water. Produced fluids from a hydrocarbon well, pipeline or formation may comprise a mixture of hydrocarbon and water. Thus, the method may involve producing a fluid comprising water and for example a mixture of hydrocarbon and water from the hydrocarbon well, pipeline or formation; removing a fluid sample from the fluid; measuring a condition parameter such as the pH of the fluid sample; adding a reagent to modify a condition parameter such as the pH of the fluid sample; and analysing the luminescence of the produced fluid to determine an amount of the nanoparticle tracer present in the fluid sample.

Produced fluids from a hydrocarbon well, pipeline or formation may comprise a mixture of an oil phase and a water phase. Typically, these phases may be separated before tracer analysis is performed. Preferably the fluid comprises a produced water phase from which the oil phase has been largely removed. Thus, the method may involve producing a fluid comprising produced water from which the oil phase has been largely removed, for example being a fluid in which the oil phase comprises no more than 10% by volume, more preferably no more than 1% by volume, and for example consisting essentially of produced water from which the oil phase has been substantially removed.

Even in a produced water phase from which the oil phase has been largely removed, water soluble organic species which are naturally fluorescent may be present in the produced water. The use of luminescent nanoparticle water tracers in accordance with the invention in a method which enhances their luminescence by adding a reagent to modify the conditions, and thus potentially improves their detectability against such background fluorescence, accordingly confers the aforementioned advantages.

In a second aspect of the invention, there is provided the use of a tracer in monitoring a fluid, wherein the tracer comprises a plurality of luminescent nanoparticles selected to undergo luminescence in an aqueous fluid that varies in the presence of various reagents/in particular conditions, and most particularly that varies with pH. More completely the invention comprises the use of such a tracer in combination with a reagent selected to be one that causes the said variation in luminescence.

A particular preferred use according to the second aspect of the invention is use in monitoring a parameter of a hydrocarbon well, pipeline or formation, and discussion herein considers such use by way of example, but other uses for example in process diagnostics and other areas where the use of a tracer or taggant composition may be encompassed within the scope of the invention

In particular, the use comprises taking a sample of a monitored fluid and modifying a condition parameter such as the pH of the sample in order to enhance detection of the presence of the tracer therein.

In particular, the use comprises application of the method of the first aspect of the invention to determine a level of the tracer in the monitored fluid.

Preferred features of the second aspect of the invention will accordingly be understood from discussion of the first aspect of the invention by analogy.

In the case where the method or use is to monitor a parameter of a hydrocarbon well or formation, the luminescent nanoparticle may be introduced into the well as a tracer by any method. For example, the introducing may comprise injecting the nanoparticle into the well or formation. For example, the nanoparticle tracer may be injected into the well or formation of which the parameter is being monitored. The nanoparticle tracer may be injected into an adjacent well or formation and thus be introduced into the formation via the adjacent well or formation. The nanoparticle tracer may be introduced into the well or formation during construction of the well. For example, the tracer may be provided comprised in a solid article incorporated into or attached to a component part of the well, such as a filter, mesh, sand screen, in-flow control device or valve. The tracer may be introduced into the well or formation as a liquid, for example in solution or as an emulsion with injection fluid, such as drilling fluids, hydraulic fracturing fluids or injection water. The tracer may be introduced into the well as a solid, for example as slurry with drilling fluids, hydraulic fracturing fluids or injection water, or as a solid or liquid encapsulated in another solid. The tracer may be introduced into the well or formation by introducing a proppant or polymer which comprises the tracer.

References to the addition of the reagent to a fluid sample should not be taken as limiting the method to batch testing of samples at a remote location. In particular, in the preferred case of application of the method to the monitoring of produced fluids from hydrocarbon wells, pipelines or formations, it should not be taken as limiting to off-line testing of samples at a remote location. Methods of analysing of fluids, and especially fluids containing tracers, in such a scenario may be divided into:

Off-line where a portion of fluid is taken away for analysis by a remote instrument;
On-line, where a sampled bypass flow is analysed by an instrument more or less in situ and is returned to the main flow;
At-line, where a sampled bypass flow is analysed by an instrument more or less in situ but is not returned to the main flow;
In-line, where the instrument directly analyses the main flow.

Based on such a definition the analysing of the luminescence of a fluid sample in accordance with the invention may be performed on-line, at-line or off-line. In the latter cases, samples of the fluid may be taken and transferred to a laboratory, either at the drilling location (at-line) or at a remote location (off-line) for analysis. Preferably the analysis is carried out using spectroscopy. An advantage of the method of the invention may be that the tracer may be readily distinguishable from prior art tracers, many of which now already contaminate a large number of hydrocarbon wells.

The analysis may be qualitative, in that it determines whether the luminescent nanoparticle is present or not; or it may be quantitative in that it determines if the luminescent nanoparticle is present by determining the level, for example the concentration, of the luminescent nanoparticle in the fluid sample; or it may be semi-quantitative in that by using the production rates it determines the relative flow from different regions of the hydrocarbon well. Preferably the analysis determines the level at which the luminescent nanoparticle is present in the fluid sample. The level may be determined as a ratio of parts of luminescent nanoparticle per part of fluid for example. Thus, the method may comprise determining the concentration of the luminescent nanoparticle in the fluid sample.

Optionally, the method in accordance with the invention may comprise the use of nanoparticles exhibiting more than one luminescence response and for example more than one wavelength of peak luminescence intensity, so long as the tracer comprises at least one set of nanoparticles exhibiting a luminescence intensity that varies in the presence of the added reagent.

Where the method of the invention comprises the use of a luminescent nanoparticle exhibiting a luminescence intensity that varies in accordance with the principles of the invention as a tracer, such use may be in conjunction with at least one other tracer or class of tracer. Such other tracer may for example be a water dispersible or oil dispersible tracer. Such other tracer may for example be an optical or luminescent tracer. Such other tracer may be a nanoparticle tracer or may be a tracer that is not a nanoparticle tracer.

For example, the other tracer can be one used for enhanced oil recovery or to monitor hydrocarbon wells, pipelines, formations.

In another example, the other tracer can be one used for any other application in process diagnostics and other areas where the use of a tracer or taggant composition may be applicable.

The other tracer may be one used to track the movement of a well treatment agent. An example well treatment agent can be a corrosion inhibitor.

Preferably luminescent nanoparticles are used in accordance with the invention as a water tracer. Preferably therefore, the nanoparticles comprising the tracer are water soluble or water dispersible. In a possible embodiment, at least a part of the surface of the nanoparticle is hydrophilic and/or oleophobic. For example, at least a part of the surface of the nanoparticle comprises hydrophilic groups, for example selected from one or more of: amine groups, hydroxyl groups, carbonyl groups. Additionally, or alternatively the outer surface may be otherwise functionalized to improve stability and/or the luminescent properties.

Techniques for modifying the surface of carbon-based nanoparticles to give such functionality are known.

Thus, the use may involve monitoring the flow and/or movement of water through or from a well or formation. For example, the use may determine the source of produced water by introducing the tracer into a defined part of the well or formation and monitoring for the presence of the tracer in produced water. As another example, the use may involve a partitioning study to determine residual oil saturation where the tracer is used as the conservative, water soluble tracer. As another example, the use may involve determining the presence or absence of a well treatment agent which had previously been tagged.

In a possible alternative application however, the tracer is an oil tracer. In such a case, the carbon-based nanoparticles comprising the tracer are soluble or dispersible in the oil phase. In a possible embodiment, at least a part of the surface of the carbon-based nanoparticles is hydrophobic and/or oleophilic. For example, at least a part of the surface of the carbon-based nanoparticles comprises hydrophobic groups and/or the surface is otherwise functionalized to improve stability and/or the luminescent properties.

Tracers of the invention may have sufficient thermal stability to survive the conditions in a hydrocarbon well. Such tracers may also be detectable, for example using GC-MS, in very low concentrations, for example concentrations of 10 ppb or less, preferably concentrations of 1 ppb or less, more preferably concentrations of 100 ppt or less, yet more preferably concentrations of 10 ppt or less and still more preferably concentrations of 1 ppt or less. The tracers may be fabricated to show a high selectivity towards water instead of oil. Thus, the tracer may be a water tracer. The tracer may have a log P value of less than −1. The log P value is a well-known value for characterising the partitioning preference of a compound for water or oil. The value is the log of the ratio of the equilibrium concentration of a species in oil (octanol) to the equilibrium concentration of the species in water. Thus, the concentration of the tracer in water is preferably at least 10 times, and more preferably at least 100 times, that of the tracer in oil.

The parameter monitored by use of the tracer may be a parameter related to a property, such as flow or composition, of the well, pipeline or formation and may be an absolute parameter or a relative parameter. A relative parameter may describe a property of one part of the well, pipeline or formation relative to another part. Examples of parameters that may be monitored include a relative distribution of water production along a lateral or between laterals in multiple interconnected well systems, a formation fluid composition, or a measure of rock heterogeneity. Preferably, the parameter relates to a well or formation. It will be appreciated that when a parameter is said to relate to a well or formation, that well refers to the constructed apparatus for extracting the hydrocarbon, while formation refers to the natural structure in which the hydrocarbon is located and from which it is extracted via the well.

It will be appreciated that features described in relation to one aspect of the invention may be equally applicable in another aspect of the invention. For example, features described in relation to the use of the tracer of the invention may be equally applicable to the method of the invention, and vice versa. The skilled person will realise where some features may not be applicable to, and may be excluded from, particular aspects of the invention.

### DESCRIPTION OF THE DRAWINGS

Embodiments of the present invention will now be described, by way of example, and not in any limitative sense, with reference to the accompanying drawings, of which:

FIG. 1 shows the typical fluorescence intensity response spectrum of produced water;

FIG. 2 shows a measured fluorescence intensity response spectrum of a known fluorescent carbon-based nanoparticle suitable for use in the method of the invention;

FIG. 3 show the difference of the fluorescence intensity response of the fluorescent carbon-based nanoparticle of FIG. 2 in a solution where the pH of the solution is approximately 4 and 9

FIG. 4 shows the variation of the peak height of the fluorescent carbon-based nanoparticle of FIG. 2 plotted against pH where the pH of the solution varies over a range from approximately 4 to 9;

FIG. 5 shows an example use of such a carbon-based nanoparticle as a tracer in a method of monitoring a hydrocarbon reservoir;

FIG. 6 shows the effect of cysteine on the fluorescence properties of nanoparticles;

FIG. 7 shows the effect of L-ascorbic acid on the fluorescence properties of nanoparticles.

### DETAILED DESCRIPTION

### Effect of pH on Fluorescent Properties of Nanoparticles

In an example of the method of the invention, fluorescent carbon-based nanoparticles are used as tracers which exhibit a fluorescence that varies with pH, and most particularly that exhibit a fluorescence that may be enhanced at a known excitation energy at a known pH, and a reagent is added to vary the pH and produce such an effect.

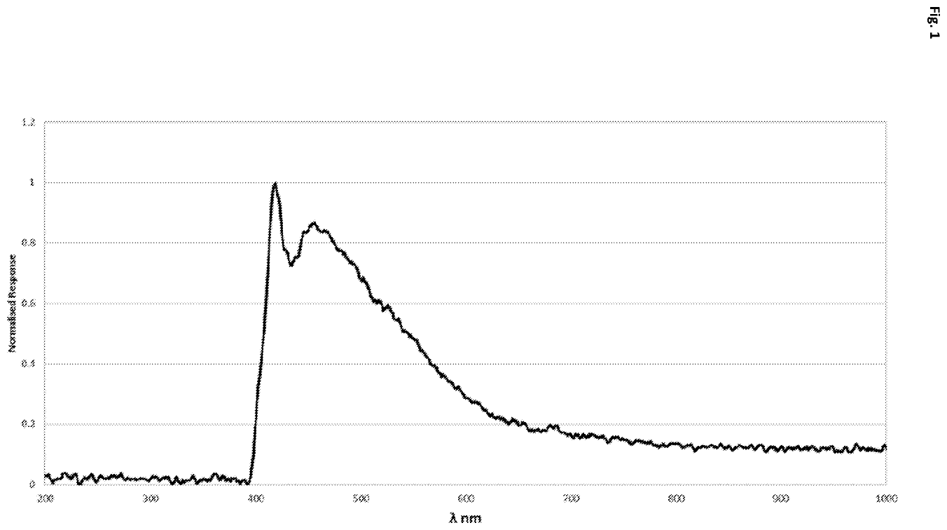

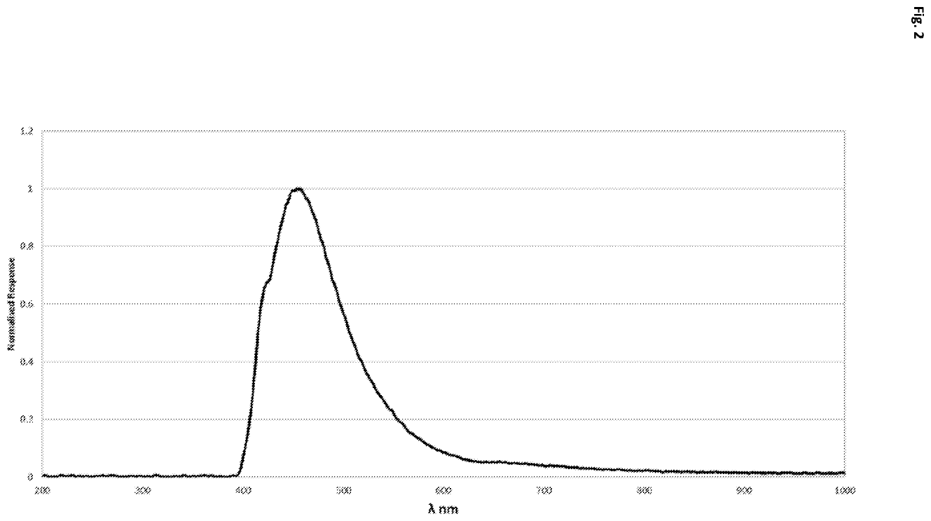

FIGS. 1 and 2 show respectively an intensity spectrum for a sample of produced water and a comparable spectrum for a known fluorescent carbon-based nanoparticle, each with normalized peak height, to illustrate the problem of background fluorescence.

FIG. 1 shows a sample of produced water from which at least 99% of the oil phase has been removed. Even so, there is strong fluorescence from organics which have distributed into and for example dissolved in the produced water phase. The peak region of fluorescence is in particular found to occur at shorter wavelengths in the visible spectrum. Only limited fluorescence is exhibited above 500 nm, even less above 550 nm, and almost none beyond 600 nm.

In FIG. 2 a comparable spectrum is shown for a known fluorescent carbon-based nanoparticle having a peak fluorescence intensity at the blue end of the visible spectrum. As can be seen, this exhibits strong fluorescence in the blue/cyan end of the spectrum, with most fluorescence occurring in the range 450-520 nm.

It can be seen that a major part of the intensity of the background fluorescence overlaps with the peak fluorescence intensity of the fluorescent carbon-based nanoparticles.

Any method that could help distinguish these responses, for example by enhancing the fluorescence response of the fluorescent carbon-based nanoparticle so as to reduce the background effect, is likely to be advantageous.

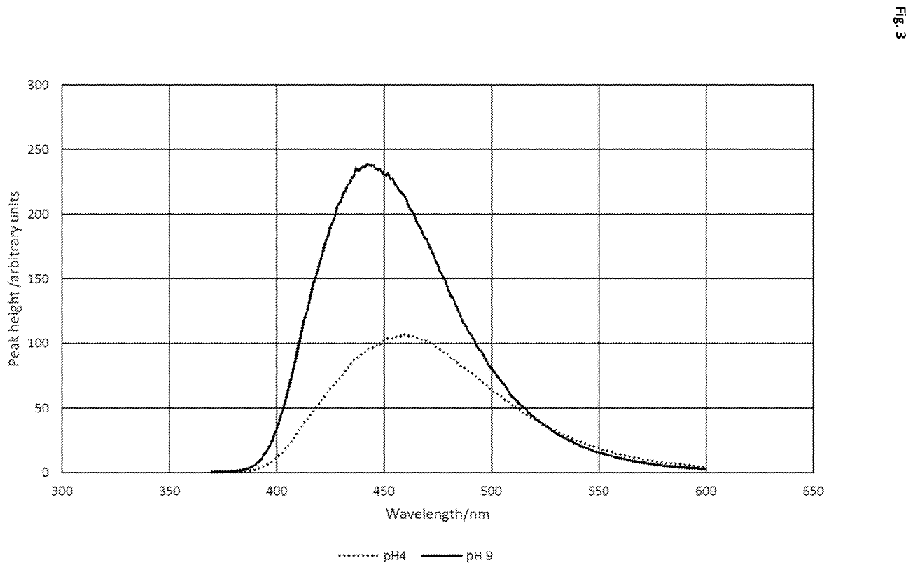

FIG. 3 show the difference of the fluorescence intensity response of the fluorescent carbon-based nanoparticle of FIG. 2 in a deionized water solution where the pH of the solution is respectively approximately 4 (broken line) and 9 (solid line).

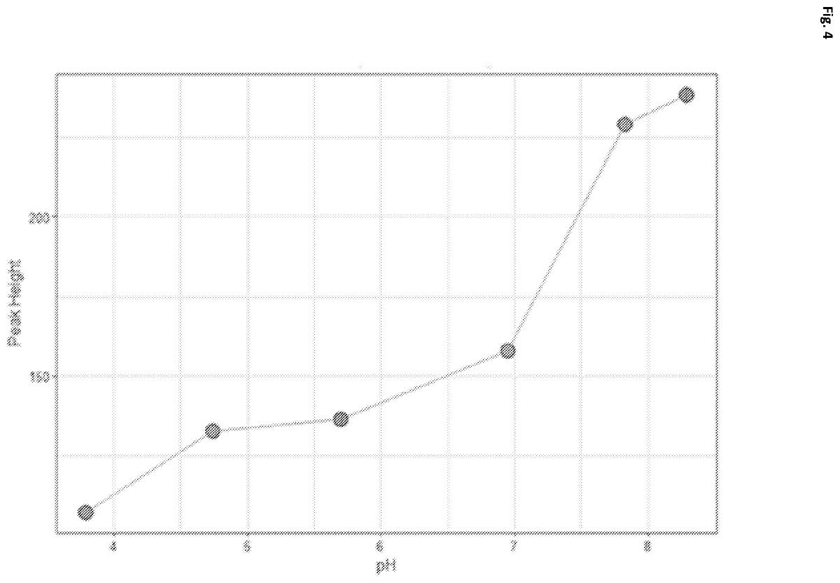

In FIG. 4, a measured fluorescence intensity peak height for the fluorescent carbon-based nanoparticle of FIG. 2 in deionized water solution is plotted against pH of that solution where the pH of the solution varies over a range from approximately 4 to 9.

The pH of the solution is varied by adding a suitable reagent to the solution. In the example given a buffer solution was used. In practical systems in the field a more powerful reagent such as NaOH may be preferred.

It can be seen that enhanced fluorescence intensity is obtained as the pH of the solution increases. The two figures show that it is possible over this range to increase the peak height by a factor of two or more by modifying the pH over the range from approximately 4 to approximately 9.

To be applicable to the invention it might be preferable that the luminescence and in the example case fluorescence of a nanoparticle tracer is at least 10% higher at the modified pH than at the unmodified pH. It follows that preferably the nanoparticle is selected to exhibit a variable luminescence and for example fluorescence response with pH that varies from a lower level of fluorescence intensity to a higher level of fluorescence intensity at least 10% higher than the said lower level of fluorescence intensity across a pH range that represents a range that can be practically modified by addition of a suitable reagent. Where the fluid is produced water from a hydrocarbon well, such a range might be for example across a pH of 5 to 9.

It can be seen that across this range in the example embodiment the fluorescence intensity varies by about a factor of two. Moreover, the carbon-based nanoparticles are found to remain chemically stable across this range. Such a combination of variation in fluorescence with chemical stability makes the carbon-based nanoparticles admirably suited for application in the method of the invention. Other nanoparticles exhibiting a similar luminescence variation across a similar pH range with good chemical stability across the range will be likely to be similarly useful for application in the method of the invention.

In practical use, the carbon-based nanoparticles are introduced as an aqueous tracer for example using a familiar technique into a hydrocarbon well, pipeline or formation; an aqueous produced fluid is obtained from the hydrocarbon well, pipeline or formation into which a proportion of the tracer has passed; a sample is taken, NaOH or another reagent with similar effects is added to modify the pH of the sample; and the modified sample is then analysed for the presence of the carbon-based nanoparticle.

The fluorescence intensity of the carbon-based nanoparticles in the sample is increased. The effect of the fluorescence attributable to the carbon-based nanoparticle tracer in the sample is enhanced relative to the background fluorescence attributable to residual organics. It becomes easier to distinguish the fluorescence of the tracer and the fluorescence of the residual organics. The effectiveness of the tracer is increased.

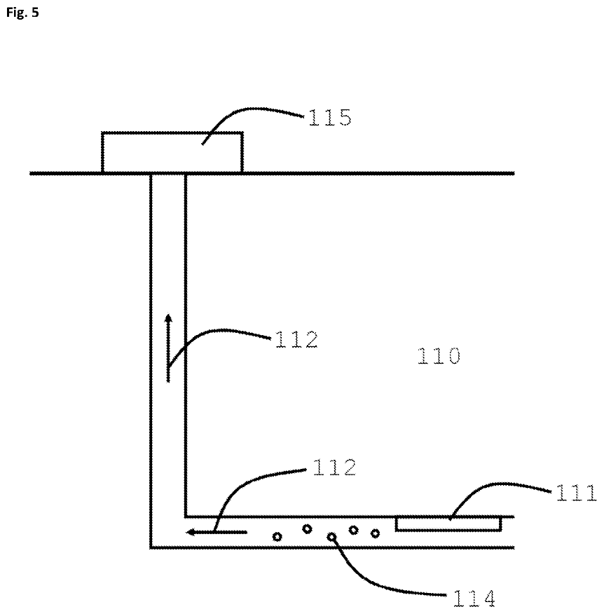

FIG. 5 provides a simple schematic of a method of monitoring a hydrocarbon reservoir 110 using such a tracer. The method comprises introducing a tracer 114 into the reservoir 110, producing fluid from the reservoir 110 and detecting the tracer 114 in the fluid so as to monitor the reservoir 110. In this example, the tracer 114 is introduced into the reservoir 110 in a release system 111. The tracer could also be injected (for example as in a water-flood application) or be there before production (for example in a hydraulic fracturing operation). The tracer 114 is released from the release system 111 and carried by the production flow 112 of the reservoir fluids to the surface where it is first treated as above to modify the pH and enhance the fluorescence effect and then detected using a suitable known apparatus and method. The reservoir 110 includes a surface facility 115 and a pH modification and a fluorescence detection apparatus is installed and the method carried out at the surface facility 115. Preferably the analysis is carried out on-line in real time.

### Example Carbon Nanoparticle Synthesis Method

30 mL of glutathione in formamide (10% w/v) was added to a sealed microwave reactor (100 mL in volume) and irradiated to maintain a temperature of 180° C. for 30 minutes. Once cooled the reaction product was added to acetone (60 mL) and cooled to 0° C. for 1 h. The mixture was then centrifugated at 10k RCF (relative centrifugal force) for 20 minutes. The liquid phase was discarded. Acetone (60 mL) was then added to the precipitated product followed by centrifugation at 10k RCF for 20 minutes. The precipitated product was then dispersed in deionised water (100 mL) and filtered through a 100 nm polyether sulfone filter.

### Effect of Cysteine on the Fluorescence Properties of Nanoparticlees

Two 10 mL solutions of carbon nanoparticle material were prepared in water. To the first solution, 0.3 g of L-cysteine was added. The solution was magnetically stirred at room temperature for 1 hour and was then filtered through a 100 nm polyether sulfone filter. The second solution was retained as a control.

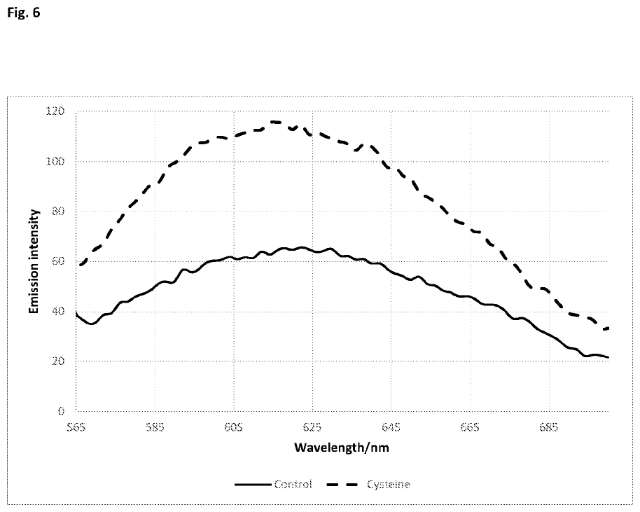

FIG. 6 shows the emission intensity of the cysteine treated product versus the control solution. As can be seen from the graph, emission intensity is significantly increased by cysteine compared to the control solution. This mechanism is distinct from the previous pH change mechanism.

### Effect of L-Ascorbic Acid on the Fluorescence Properties of Nanoparticles

A stock solution of carbon nanoparticle material in water was prepared (Control). Further solutions were prepared as follows.

To 10 mL of the stock solution, 2M hydrochloric was added to change the pH to 4-5 (Acidified(HCl)).

To 10 mL of the stock solution, 0.3 g L-ascorbic acid was added, and the solution magnetically stirred for 30 minutes. The pH of this solution was 4-5 (Acidified(Ascorbic acid)).

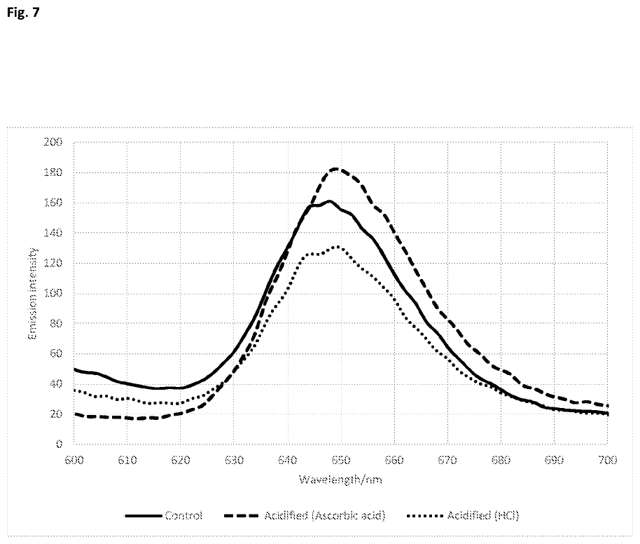

FIG. 7 shows the emission intensity of the control and the two treated product solutions. As can be seen from the graph, ascorbic acid causes an increase in emission intensity between 600 and 700 nm compared to the control. However, this effect is not related to the pH change resulting from addition of the ascorbic acid. Causing a corresponding pH change using hydrochloric acid results in a decrease in emission intensity between 600 and 700 nm compared to the control. This is consistent with the graph shown in FIG. 4 which indicates a decrease in emission with a decrease in pH. As such, the increase in emission intensity with addition of ascorbic acid in this example is due to another mechanism distinct from pH change.

### CONCLUSIONS

It is apparent that there are several different reagents and mechanisms which can be used to vary the luminescence behaviour of luminescent nanoparticles and/or of other luminescent species present in a fluid sample in order to more readily detect the luminescent nanoparticles in a fluid sample and determine the amount of nanoparticles present. While this invention has been particularly shown and described with reference to certain embodiments, it will be understood to those skilled in the art that various changes in form and detail may be made without departing from the scope of the invention as defined by the appended claims.

1. (canceled)

2. A method of monitoring a parameter of a hydrocarbon well, pipeline or formation, the method comprising:
introducing a plurality of luminescent nanoparticles as a tracer into the hydrocarbon well, pipeline or formation;
producing an aqueous fluid from the hydrocarbon well, pipeline or formation, the aqueous fluid comprising water soluble organic species which are naturally fluorescent and the luminescent nanoparticles;
removing a fluid sample from the aqueous fluid;
adding a reagent to the fluid sample to vary the luminescence behaviour of the luminescent nanoparticles or of the water soluble species present in the fluid, wherein the reagent is selected to act either to suppress the luminescence of the water soluble organic species or to increase the luminescence of the luminescent nanoparticles to enable better resolution of the luminescence of the luminescent nanoparticles from background luminescence of the water soluble organic species;
analysing the luminescence of the modified fluid sample to determine an amount of the nanoparticle present therein; and
monitoring a parameter of the hydrocarbon well, pipeline or formation based on the determined mount of nanoparticles present in the modified fluid sample, wherein the parameter being monitored is the flow of water through or from the hydrocarbon well, pipeline or formation.

3. (canceled)

4. A method according to claim 1, wherein the aqueous fluid comprises produced water from which the oil phase has been largely removed.

5. (canceled)

6. A method according to claim 1 comprising adding the reagent at least to vary the luminescence of the luminescent nanoparticles.

7. A method according to claim 3 comprising selecting a combination of luminescent nanoparticles and reagent that are known to interact together in aqueous solution such that the luminescence behaviour of the luminescent nanoparticles varies in the presence of the reagent.

8. A method according to claim 1, wherein the reagent is selected to change a condition parameter of the fluid, the said condition parameter being one the variation of which is known to cause a variation in luminescence of the luminescent nanoparticles or of the water soluble organic species present in the fluid.

9. A method according to claim 5 comprising the additional step of measuring a condition parameter of the fluid sample before adding the reagent, and subsequently adding the reagent to change the condition parameter.

10. A method according to claim 5, wherein the condition parameter is the pH of the aqueous fluid.

11. (canceled)

12. (canceled)

13. (canceled)

14. A method according to claim 1, wherein the luminescent nanoparticles are selected to have a surface functionality modified such as to cause the nanoparticle to exhibit a luminescence response that varies with varying pH.

15. A method according to claim 8, wherein the surface of the luminescent nanoparticles comprises one or more functional groups that act in aqueous solution as proton donors or proton acceptors.

16. A method according to claim 8, wherein the surface of the luminescent nanoparticles comprises one or more functional groups selected from: carboxyl, carbonyl, sulfonyl, hydroxyl, thiol, amine, amide, imide, and combinations and derivatives of the same.

17. A method according to claim 1, wherein the luminescent nanoparticles comprise carbon-based nanoparticles.

18. A method according to claim 1, wherein the luminescent nanoparticles are doped.

19. A method according to claim 1, wherein the luminescent nanoparticles are water-dispersible.

20. (canceled)

21. A method according to claim 1, wherein at least a part of the surface of the luminescent nanoparticles comprises hydrophilic groups, for example selected from one or more of: amine groups, hydroxyl groups, carbonyl groups.

22. A method according to claim 1, wherein the luminescent nanoparticles are fluorescent nanoparticles.

23. (canceled)

24. (canceled)

25. (canceled)

26. (canceled)

27. (canceled)

28. (canceled)
